(19) United States
(12) Patent Application Publication
(10) Pub. No.: US 2010/0154800 A1
(43) Pub. Date: Jun. 24, 2010

(54) ENDOTRACHEAL TUBE WITH SECURE COLOR-CODED CONNECTOR AND PACKAGING

(76) Inventors: Chai Jie Chang, Modesto, CA (US); John Duval, Long Beach, CA (US); Frank Segarra, Merced, CA (US); Tamim Wafa, Modesto, CA (US)

Correspondence Address: LAW OFFICES OF DAVID M. LANG, 3051 Daggett Drive, Granite Bay, CA, 95746, US

(21) Appl. No.: 12/485,908

(22) Filed: Jun. 16, 2009

Related U.S. Application Data
(60) Provisional application No. 61/073,165, filed on Jun. 17, 2008.

(51) Int. Cl. A61M 16/04

(52) U.S. Cl. 128/207.15

(57) ABSTRACT

An endotracheal tube system having an improved connector to provide a more secure connection to an endotracheal tube. In specific example embodiments, the connector is tapered, is spiraled, or provides threading to improve connection and prevent inadvertent detachment. In certain embodiments, the connector is colorized to correspond to an endotracheal tube size, and in an embodiment, specific packaging permits color to be visualized to aid in proper tube size selection.

### CROSS-REFERENCE TO RELATED APPLICATIONS

This application claims the benefit under 35 U.S.C. §119(e) to U.S. Provisional Application U.S. 61/073,165 entitled “Endotracheal tube with color-coded secure connector and packaging” filed Jun. 17, 2008, which is herein incorporated by reference as if more fully set forth.

### FIELD OF THE INVENTION

The present invention relates to medical devices generally. More specifically, the invention relates to an improved endotracheal tube connector, as well as a packaging, and sizing system.

### BACKGROUND

Endotracheal intubation is a necessary skill for a variety of medical professionals. Anesthesiologists, Emergency Physicians, Paramedics, and other professional personnel secure a patient's airway by placement of an endotracheal tube (also known as an ET tube) within the trachea of a patient. Placement may occur in a controlled way, such as an operating room environment, or may be undertaken more emergently at bedside, in the Emergency Department, or by trained ambulance personnel in a field or battlefield setting. Proper placement of an endotracheal tube is of paramount importance; potentially catastrophic implications may be expected where a patient is not ventilated or is inadequately ventilated.

Endotracheal tube placement may be difficult and challenging, even for seasoned professionals, and may be complicated by the patient's physiological status, as well as other factors, including traumatic deformation of the oropharynx, blood, dentition, vomitus, or other foreign material in the patient's oral cavity. Additionally, infants and pediatric patients may present special challenges in securing adequate tube placement.

Following successful tube placement, professionals are cautious to avoid disconnecting the connector from proximal portion of the endotracheal tube; unrecognized disconnections may be life threatening. The probability that a connector will become unintentionally disconnected is increased where ventilation occurs in a setting outside the operating room, such as a complicated field setting (e.g. the extrication of a trapped patient), or where the patient must be moved. Accordingly, it is important to maintain proper tube placement—particular in circumstances where initial placement was difficult.

Selecting the proper size ET tube size is also critically important. Where the chosen tube is too small, the relatively smaller cross-sectional luminal diameter will be smaller compared to the required tidal volume—making ventilation more difficult. Further, an improperly small tube may be more subject to dislodgement, may inadequately seal the airway, and may make any necessary subsequent required instrumentation occurring through the tube (e.g. bronchoscopy) more difficult. In contrast, an improperly large tube may be unnecessarily difficult to pass and cause trauma to the airway. Improper tube selection is more common under rushed or emergent circumstances, where the professional performing the skill has relatively limited recent experience, and in settings where intubation is infrequently performed.

The need to verify proper tube placement and size is common, particularly where a patient is transferred from one professional or service to another. As a placed tube is often secured by tape, confirmation of the tube size may be difficult where tube sizing indicia itself is obscured by the tape. Lastly, patient management may become problematic where the free end of the pilot balloon line becomes entangled in other equipment or where the pilot balloon cannot be quickly located where intubation is performed in unfavorable field settings.

There have been devices utilizing color coding to assist with identification of tube size. For example, U.S. Pat. No. 4,483,337 to Clair, discloses, in part, a color-coding system for identifying endotracheal tube size, including a color-coded strip along the longitudinal length of the tube from the connector to approximately the area of the exit of the pilot line. U.S. Pat. No. 6,050,263 to Choksi, et al. discloses, in part, an endotracheal tube holder having, inter alia, a support bar and platform and tube that may have coloration to indicate size. U.S. Pat. No. 4,926,885 to Hinkle discloses, in part, a method of identifying and selecting medical equipment, medication doses and/or medical supplies, and to the equipment used to carry out the method; the method is largely based upon anatomical dimensions. However, the prior art solutions are not without problems. For example, use of a color coded strip may interfere with patient care efforts and become soiled in any traumatic resuscitative events. Apparatus designed to serve as an endotracheal tube holder may be cumbersome in certain clinical circumstances and require specific clinician familiarity before use. Further, methodologies derived largely from anatomical dimensions may be more time consuming to apply, particularly in emergency situations.

### SUMMARY

The present invention discloses a color-coding system which matches the color of an endotracheal tube and connector to a specific cross-sectional luminal diameter. Further embodiments provide for corresponding packaging permitting at least a portion of a colored connector, tube, or both to be visible through the packaging thereby aiding in rapid selection of an appropriate tube size. The invention further discloses a series of non-uniform improved endotracheal tube connectors to provide a more secure attachment between an endotracheal tube and connector and may permit the connector to be secured to a tube more tightly than a conventional connector. Further, the connector may be tightened more easily as the user applies a twisting action to insert the connector into the tube. Further, in specific embodiments, the connector has threading which may provide a more secure connection by disrupting a fluid layer that may accumulate between the connector and tube's interior. Further, as at least a portion the tube must stretch or deform to accommodate certain embodiment connectors, the tube must likewise change shape before it can slip—making inadvertent disconnection less likely. The inventive connector will allow purposeful separation when needed. In a further embodiment, the connector bears one or more notches where the pilot balloon of an endotracheal tube may be secured.

### BRIEF DESCRIPTION OF THE DRAWINGS

FIG. 1 is a perspective view of an embodiment endotracheal tube.

FIG. 2 is a perspective view of an embodiment endotracheal tube illustrating the pilot balloon line secured to the connector.

FIG. 3 is a perspective view of an embodiment endotracheal tube connector.

FIG. 4 is a perspective view of an embodiment endotracheal tube connector.

FIG. 5 is a chart showing an illustrative example embodiment color-coding system.

FIG. 6 is a perspective view of example embodiment packaging.

FIG. 7 is a perspective view of example embodiment packaging.

FIG. 8 is a perspective view of an embodiment barb connector.

FIG. 9 is a perspective view of an embodiment leading spiral connector.

FIG. 10 is a perspective view of an embodiment trailing spiral connector.

FIG. 11 is a perspective view of an embodiment negative spiral connector.

FIG. 12 is an elevation view of an embodiment positive spiral connector.

FIG. 13 is an elevation view of an embodiment tapered diamond thread spiral connector.

FIG. 14 is an elevation view of an embodiment tapered shaft, diminishing bullnose thread connector.

FIG. 15 is an elevation view of an embodiment tapered shaft, non-diminishing bullnose thread connector.

FIG. 16 is an elevation view of an embodiment non-tapered shaft, non-diminishing flattened diamond thread connector.

FIG. 17 is an elevation view of an embodiment non-tapered shaft, diminishing flattened diamond thread connector.

### DETAILED DESCRIPTION

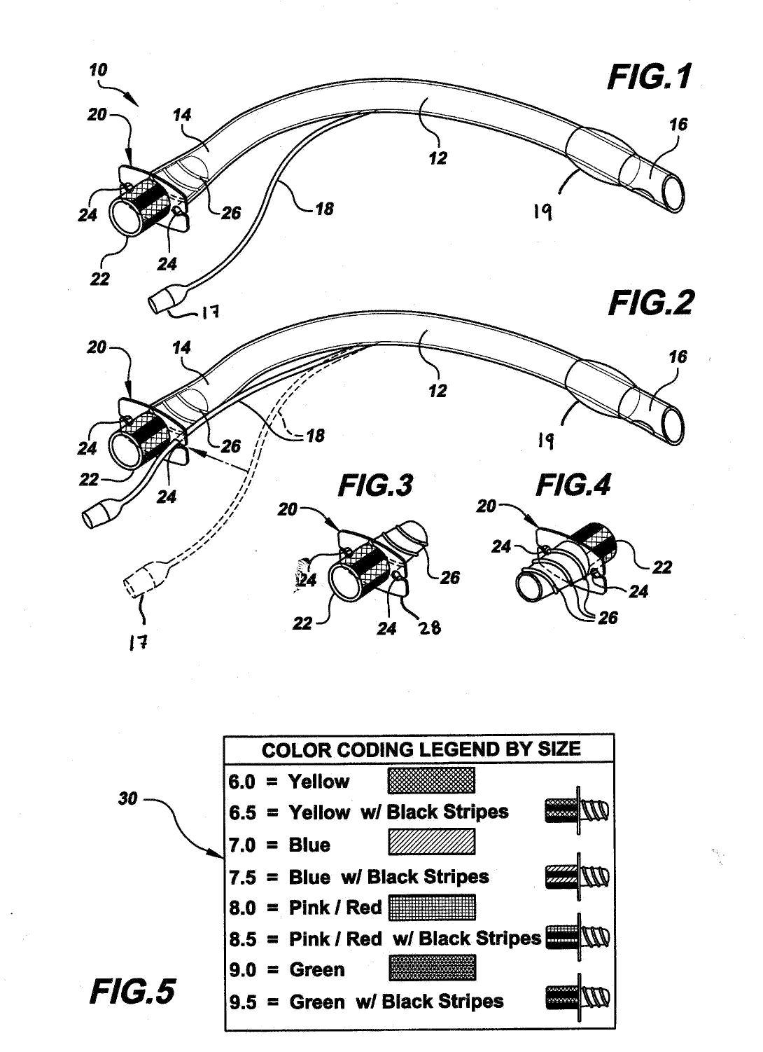

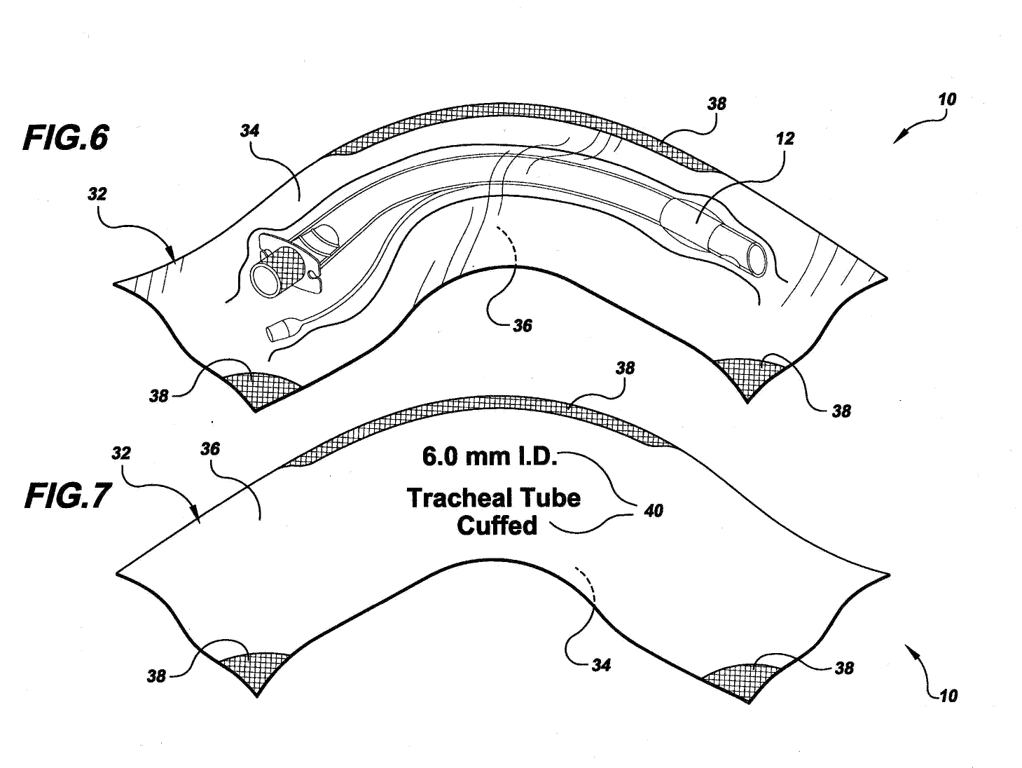

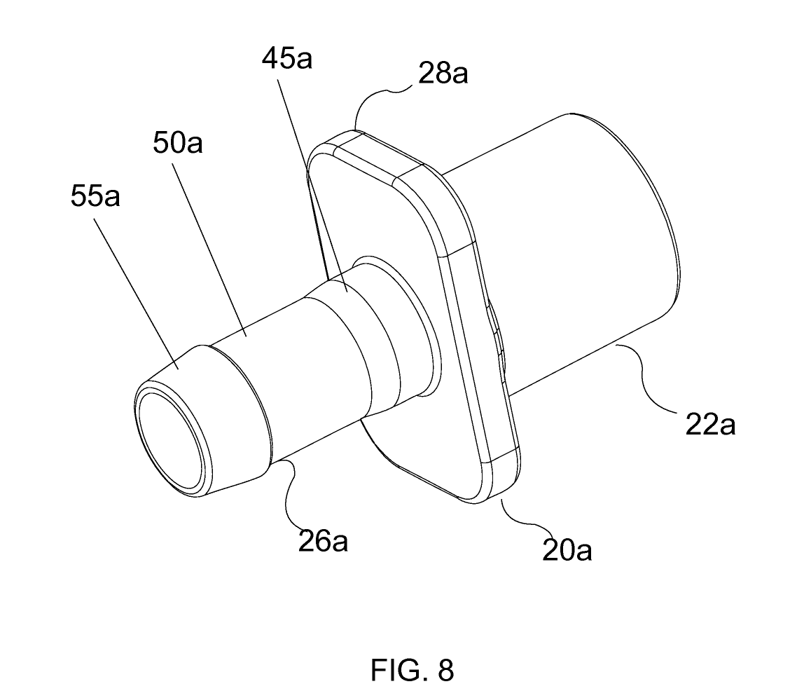

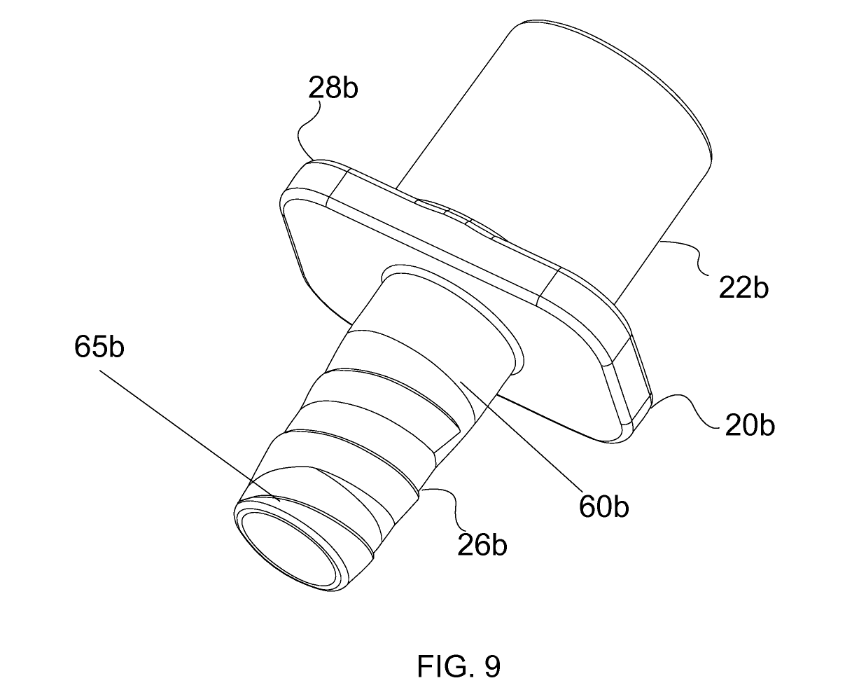

In one embodiment of the present invention, FIG. 1 illustrates an endotracheal tube generally 10 comprising a tube body 12 commonly comprised of PVC tubing having a proximal end 14 and a distal end 16. There is an air instillation port 17 communicating with a pilot balloon and pilot balloon line 18 which leads to cuff 19 located relatively distally on tube body 12. In one embodiment, illustrated by FIGS. 3 and 4, connector 20 is comprised of a first end 22 that is uniform in diameter, which slides within and reversibly affixes to a ventilation device such as a mechanical ventilator, the Y-piece of an anesthetic delivery system, or a conventional bag-valve combination, such as an Ambu® bag. The second end 26 connects to an endotracheal tube. Second end 26 of connector 20 is coupled to the proximal end 14 of tube body 12. A planar surface 28 between first end 22 and second end 26 aids in providing a space to hold and manipulate the connector 20 as well as tube 12 when connector 20 is coupled thereto. In one embodiment, connector 20 bears notches 24 on one or both lateral sides of planar surface 28; the notches are capable of reversibly receiving and holding pilot balloon line 18 as illustrated by FIG. 2.

One aspect of the present invention is to provide an endotracheal tube connector that more securely attaches to the proximal end 14 of tube 12. Generally, endotracheal tube connectors are straight and smooth and may slightly narrowingly taper. One aspect of the present invention is to provide a series of improved connectors that permit greater resistance to dislodging from tube 12 and, in some embodiments, may prevent leakage and may decrease contamination passing around second end 26 into tube 12 or from within tube 12 around second end 26 to contaminate the outside of tube 12.

For illustrative purposes, when referring to connector 20, it will be helpful to consider the first end 22 as the proximal end and second end 26 as the distal end. In one embodiment illustrated by FIG. 8, a barbed connector 20a has a first end 22a, a second end 26a and planar surface 28a. In this embodiment, second end 26a has a flared portion 45a, which flares outwardly as second end 26a contacts planar surface 28a. Second end 26a has straight portion 50a, and terminates in barb 55a at the distal portion of the second end 26a.

FIG. 9, illustrates an alternative leading spiral embodiment, wherein a leading spiral connector 20b has first end 22b, and second end 26b, and planar surface 28b therebetween. Second end 26b has a flared portion 60b, which outwardly flares as second end 26b contacts planar surface 28b. The surface of second end 26b has a relatively broad, constant diameter spiral 65b which begins at the distal end of second end 26b. Spiral 65b terminates at flared portion 60b.

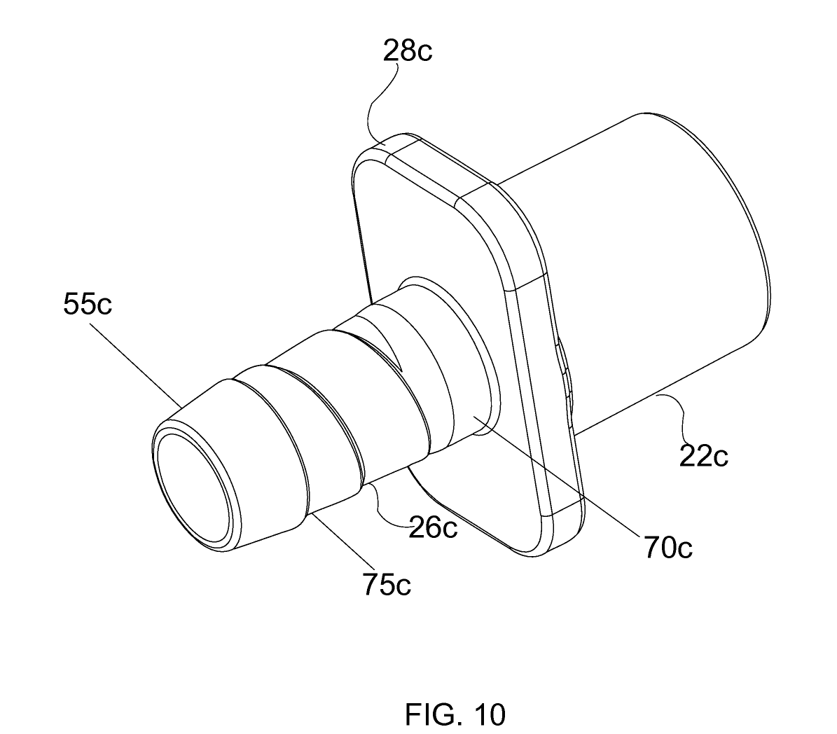

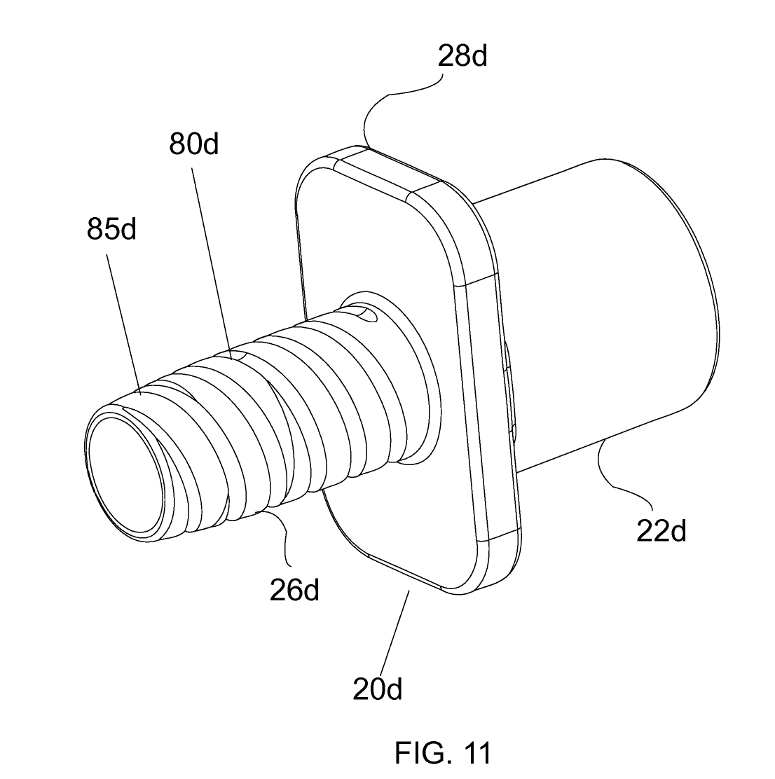

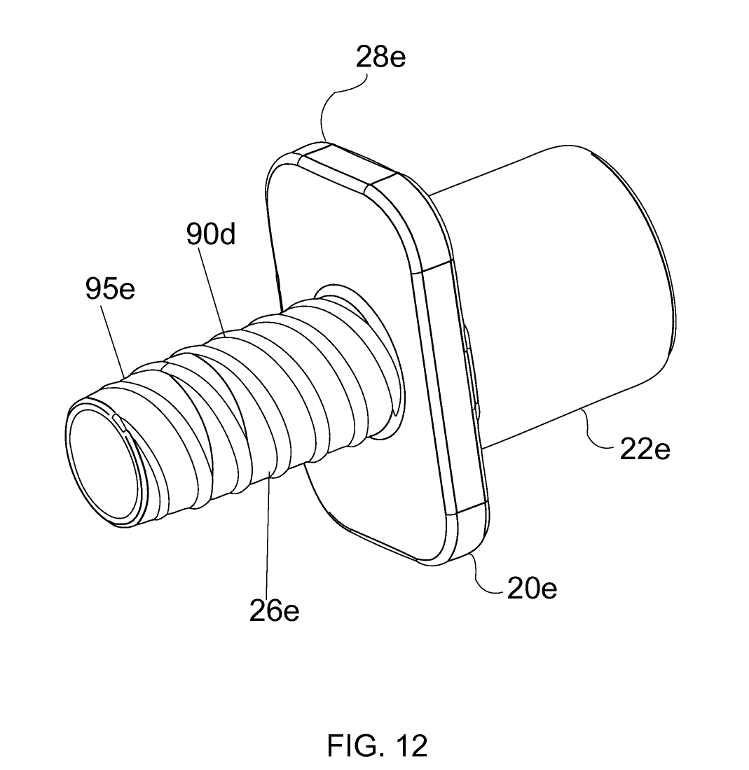

FIG. 10 illustrates a trailing spiral alternative embodiment 20c, having first end 22c, second end 26c, and planar surface 28c therebetween. Second end 26c has flared portion 70c which outwardly flares as second end 26c contacts surface 28c. Second end 26c has a tapered barb 55c, which narrowingly tapers from proximal to distal. Barb 55c is uninterrupted and creates a seal with proximal end 14 of tube 12. Flared portion 70c, creates a redundant seal where connector 20c engages the proximal end 14 of tube 12. Spiral 75c begins just proximal to barb 55c and occupies the surface of second end 26c from barb 75c to flared portion 70c.

FIG. 11 illustrates a negative spiral alternative embodiment 20d, has first end 22d, second end 26d, and planar surface 28d therebetween. The second end 26d has grooving which forms a negative spiral 80d, which narrowingly tapers to terminate at end 85d.

FIG. 12 illustrates a positive spiral alternative embodiment 20e, has first end 22e, second end 26e, and planar surface 28e therebetween. The second end 26e has a positive spiral 90e which forms a ridge along the surface of second end 26e, and narrowingly tapers to terminate at end 95e.

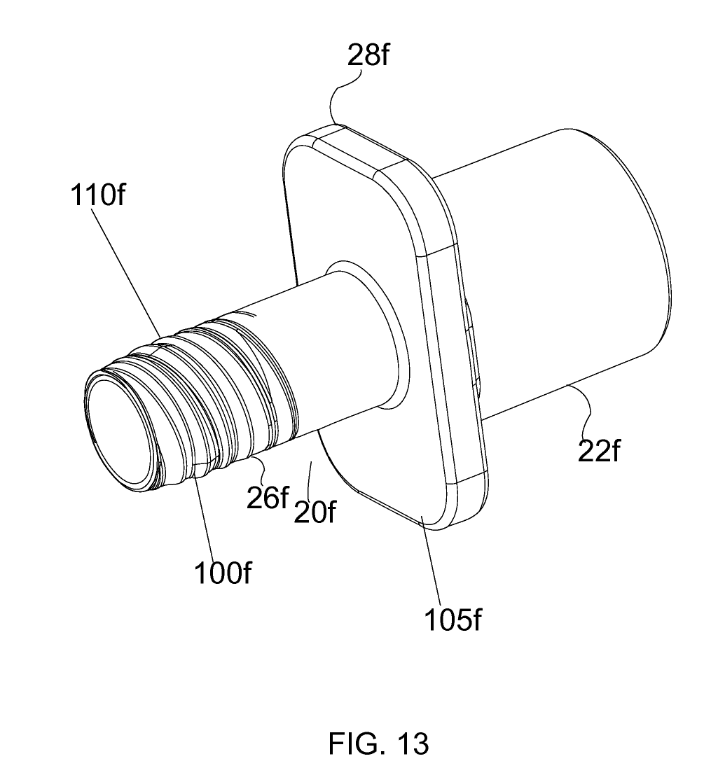

FIG. 13 illustrates a tapered spiral embodiment 20f, has first end 22f, second end 26f, and planar surface 28f therebetween. Second end 26f has a triple spiral diamond shaped spiral thread form 100f which terminates in smooth sealing surface 105f. Second end 26f begins to taper at tapering point 110f to narrowingly taper over approximately its distalmost quarter. In an alternative embodiment, second end 26 may be non-tapered, begin to taper at a different point, or taper over the entire second end 26.

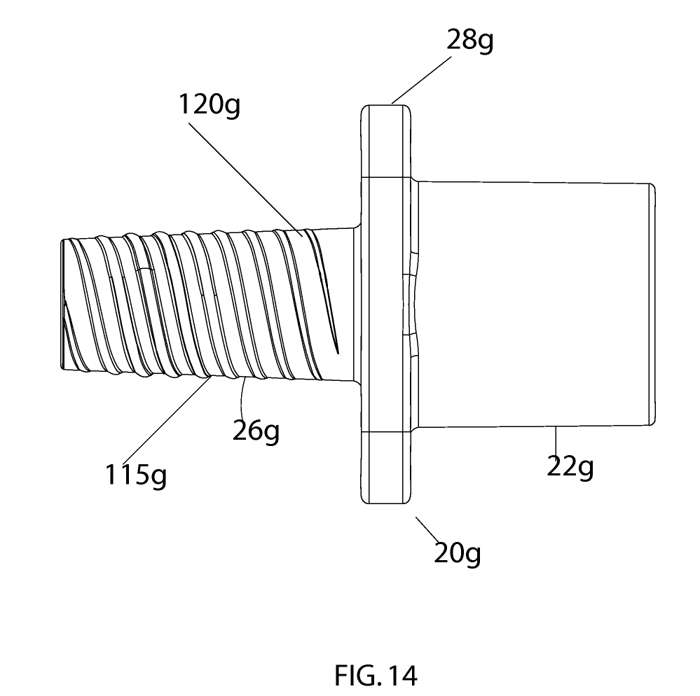

FIG. 14 illustrates a tapered diminishing thread embodiment 20g, has first end 22g, second end 26g, and planar surface 28g therebetween. Second end 26g has a bullnose thread 115g on its surface which progressively diminishes in height relative to the surface of second end 26g beginning at about the proximalmost third 120g of second end 26g. In an alternative embodiment, the thread may diminish continually over the course of second end.

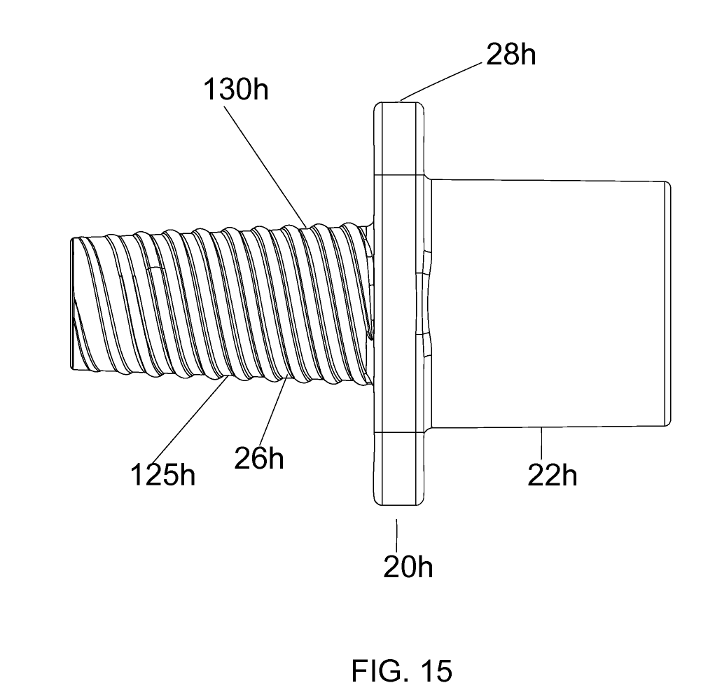

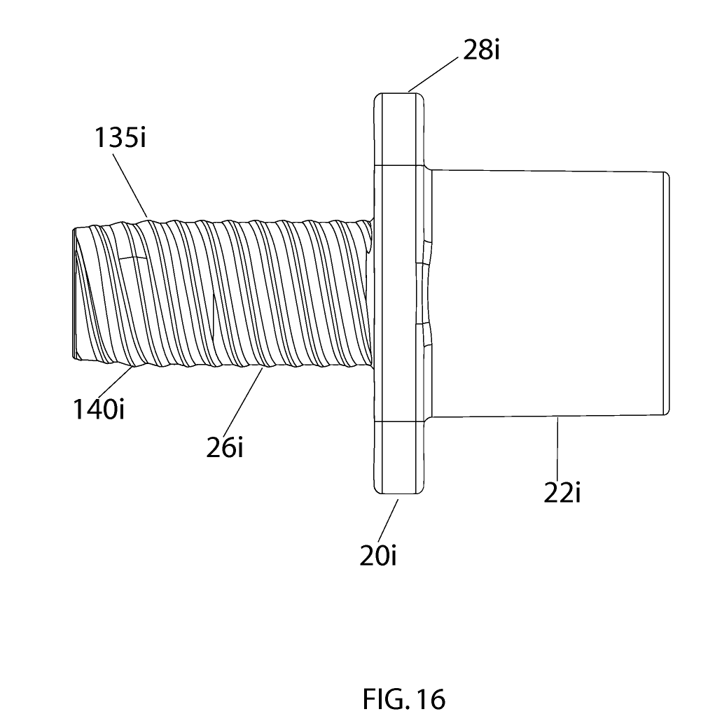

FIG. 15 illustrates a tapering, non-diminishing bullnose thread embodiment 20h, having a first end 22h, second end 26h, and planar surface 28h therebetween. Second surface 26h narrowingly tapers over the course of its entire length. Bullnose thread 125h occupies the entire surface of second end 26h.

FIG. 16 illustrates a non-tapering, non-diminishing thread embodiment 20i, having a first end 22i, second end 26i, and planar surface 28i therebetween. Second end 26i is uniform in diameter and has a flattened diamond-form thread 135i along its entire surface. The cross-section of diamond thread 135i forms a pointed apex 140i.

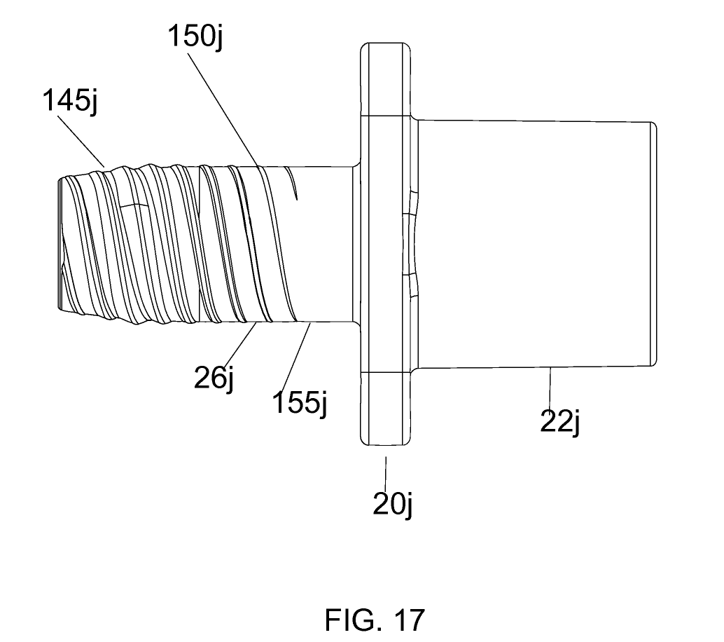

FIG. 17 illustrates a non-tapering shaft, with a diminishing diamond thread form embodiment 20j is disclosed and has first end 22j, second end 26j, and planar surface 28j therebetween. Second surface 26j has a flattened diamond-form thread 145j which progressively diminishes in height relative to the surface of second end 26j beginning at about the proximalmost third 150j of second end 26j.

It should be clear from the foregoing disclosure and expressly noted that the above example embodiments are for illustrative purposes only. It is specifically recognized that various disclosed variables may be interchanged and utilized together or independently, including: taper or non-tapering, the degree or rapidity of taper, the location for the beginning or termination of tapering (e.g. entire distance of second end 26, distal third, etc.), the presence or absence of a flaring portion (and the degree of flare), and the presence or absence of a barb. Also, multiple threading types are illustratively disclosed in non-limiting examples, such as a positive threading ridge and a negative threading groove. Threading size (including height from the surface and width across the surface of second end 26) and shape and number of windings comprising the spiral is variable as is the utilization of diminishing or non-diminishing threading. Additionally, any portion of the second end 26 may be smooth and utilized in conjunction with various thread forms. Also, as second end 26 may have a helical or spiral shape, the density, pitch, height, and width of threading on second end 26 is variable. In a specific alternative embodiment, second end 26 has a plurality of helices independent of each other.

Turning now to coloration, in one example embodiment, the endotracheal tube is color coded to correspond to a particular endotracheal tube size. Several coloration schemes can be adopted and utilized. One particular useful scheme uses solid colors for each integer size, and a color with stripes for each next non-integer half-size. An exemplar scheme, illustrated by FIG. 5, depicts a 6.0 tube as yellow, and a 6.5 tube as yellow with black stripes; a 7.0 tube as blue, and 7.5 tube as blue with black stripes; an 8.0 tube as red, and an 8.5 tube as red with black stripes; a 9.0 tube as green, and a 9.5 tube as green with black stripes. The color of connector 20 matches tube coloration; this provides easy observational recognition and/or confirmation of tube size after placement, without requiring professionals to read printed indicia on the side of tube body 12. Of course, any non-solid color can be used to indentify non-integer half-sizes. Alternatively, non-solid colors may be used to identify integer sizes. Alternatively, the sizes of tube 10 may correspond with a distinct solid or non-solid tube coloration representing a specific cross-sectional luminal size.

To facilitate easy recognition prior to use, the endotracheal tube 10 is packaged in packaging 32 which, in one embodiment comprises a first side 36 having printable paper surface that provides printed indicia 40, such as tube size information. In an alternative embodiment, printed indicia 40 may be omitted. The second side 34 has a clear transparent packaging surface allowing direct visualization of the colored tube 10 and connector 20 within packaging 32. In an alternative embodiment, the second side may have any surface that permits at least a portion of tube 10 or connector 20 to be visualized sufficient to identify the coloration of either or both. As examples, such surfaces may include a translucent surface or fenestrated surface sufficient to permit color identification.

In one specific embodiment, the coloration of the packaging is correlated to the tube size and matches the coloration of connector 20, tube 10, or both. In this embodiment, packaging 32 may be opaque.

It should be clearly understood that aspects of the present invention may be practiced independently of one another. For example, tube 10 may be colorized and connector 20 may or may not be colorized, and if connector 20 is colorized, the coloration may correspond to the coloration of tube 10 or may be distinct. Further, connector 20 may be colorized and tube 10 may or may not be colorized, and if tube 10 is colorized, the coloration may correspond to the coloration of connector 20 or may be distinct. Moreover, it should be noted that the varied embodiments of connector 20 may be utilized with or without notches 24. It should be apparent that connector 20 may be utilized with or without regard to connector or tube 10 coloration.

While exemplar tube sizes are disclosed, it is recognized that a veterinary application may result in utilization of tubes with different cross-sectional luminal diameters. Accordingly, tube size discussed herein is for illustrative purposes only, and aspects of the present invention may be utilized with larger or smaller tubes.

Although the present invention has been described with reference to the preferred embodiments, it should be understood that various modifications and variations can be easily made by those skilled in the art without departing from the scope and spirit of the invention. Accordingly, the foregoing disclosure should be interpreted as illustrative only and is not to be interpreted in a limiting sense. It is further intended that any other embodiments of the present invention that result from any changes in application or method of use or operation, method of manufacture, shape, size, or material which are not specified within the detailed written description or illustrations contained herein yet are considered apparent or obvious to one skilled in the art are within the scope of the present invention.

We claim:

1. An endotracheal tube system comprising: a tube having a tube body with a proximal end and distal end; a connector having a first end, a second end, and a planar surface, the second end coupled to the proximal end of said tube; wherein the first end of said connector is adapted to receive ventilation equipment; wherein the second end of said connector has a non-uniform shape.

2. The system according to claim 1, further comprising a cuff mounted on the distal third of said tube proximal to said opening; a pilot balloon line having a proximal end and distal end, said line coupled to said cuff at the line's distal end; a pilot balloon coupled to the proximal end of said pilot balloon line; an air instillation port coupled to said pilot balloon; wherein said tube has at least one opening on its circumferential surface distal to said cuff; wherein air may be introduced through said air instillation port, pass through said pilot balloon line to inflate said cuff, wherein said pilot balloon will become inflated after a sufficient pressure has been introduced into said cuff; wherein said non-uniform shaped connector has coloration corresponding to a specific cross-sectional luminal diameter.

3. The system according to claim 2, wherein said connector's second end has a bullnose threading on its surface.

4. The system according to claim 2, wherein said second end has a flared portion, which flares outwardly as second end contacts planar surface, the second end having a straight portion in the relative center of said second end, said second end terminating in a barb at the distal portion of the second end.

5. The system according to claim 2, wherein said second surface is spiraled.

6. The system according to claim 5, wherein said spiral is a negative spiral.

7. The system according to claim 5, wherein said spiral progressively diminishes in height relative to the surface of the second end beginning at about the proximalmost third of the second end.

8. The system according to claim 1, wherein second end narrowingly tapers over its distalmost half, and wherein said distalmost half has a triple spiral diamond shaped threading on its surface.

9. The system according to claim 1, wherein said non-uniform second end narrowingly tapers over the course of its entire length; and wherein the second end has a bullnose threading on its surface.

10. The system according to claim 1, wherein said connector has coloration corresponding to a specific cross-sectional luminal diameter.

11. The system according to claim 10, further comprising a packaging, wherein the tube is disposed within said packaging, wherein at least a portion of the tube or connector is visible through said packaging; wherein said tube has coloration corresponding to a specific cross-sectional luminal diameter; wherein coloration is visible through said packaging; wherein the perimeter of the connector's planar surface is shaped to define notches capable of receiving pilot balloon tubing.

12. The system according to claim 11, wherein said packaging is comprised of at least one transparent surface.

13. The system according to claim 1, wherein said tube and connector coloration is solid and wherein said cross-sectional luminal diameter is an integer.

14. The system according to claim 1, wherein said tube and connector coloration is not solid and wherein said cross-sectional luminal diameter is a non-integer.

15. The system according to claim 4, wherein the luminal diameter is 6.0 mm and the tube coloration is yellow; wherein the luminal diameter is 7.0 mm and the tube coloration is blue; wherein the luminal diameter is 8.0 mm and the tube coloration is red; wherein the luminal diameter is 9.0 mm and the tube coloration is green; wherein the luminal diameter is 6.5 mm and the tube coloration is yellow with black stripes; wherein the luminal diameter is 7.5 mm and the tube coloration is blue with black stripes; wherein the luminal diameter is 8.5 mm and the tube coloration is red with black stripes; wherein the luminal diameter is 9.5 mm and the tube coloration is green with black stripes.

16. The system according to claim 1, further comprising packaging having a coloration corresponding to the tube's cross-sectional luminal diameter.

17. The system according to claim 2, further comprising packaging said packaging having a coloration that is the same as said connector's coloration.

18. An endotracheal tube system comprising:
a plurality of eight endotracheal tubes, wherein each tube of said plurality has coloration corresponding to a specific cross-sectional luminal diameter;
a packaging having a first surface and second surface, wherein one of each eight tubes of the plurality is disposed within said packaging, wherein at least one surface of said packaging is transparent, wherein at least a portion of the coloration of the tube or connector of said plurality is visible through said packaging;
an endotracheal tube connector having coloration corresponding to a specific cross-sectional luminal diameter; wherein said connector is coupled to one end of said endotracheal tube, said connector having a first end and second end and a planar surface, wherein said second end narrowingly tapers from proximal to distal, the surface of said tapered second end having a bullnose shaped spiral ridge, wherein the perimeter of said planar surface is shaped to define notches capable of receiving the tubing of a pilot balloon;
wherein the tube's luminal diameter is 6.0 mm and the coloration is yellow; wherein the tube's luminal diameter is 6.5 mm and the coloration is yellow with black stripes; wherein the tube's luminal diameter is 7.0 mm and the coloration is blue; wherein the tube's luminal diameter is 7.5 mm and the coloration is blue with black stripes; wherein the tube's luminal diameter is 8.0 mm and the coloration is red; wherein the tube's luminal diameter is 8.5 mm and the coloration is red with black stripes; wherein the tube's luminal diameter is 9.0 mm and the coloration is green; wherein the tube's luminal diameter is 9.5 mm and the coloration is green with black stripes.
